(19) United States
(12) Patent Application Publication
(10) Pub. No.: US 2014/0206596 A1
(43) Pub. Date: Jul. 24, 2014

(54) Design of pH-Sensitive Oligopeptide Complexes For Drug Release Under Mildly Acidic Conditions

(71) Applicant: University of Southern California, Los Angeles, CA (US)

(72) Inventors: Wei-Chiang SHEN, San Marino, CA (US); Jennica KRANKEL, Studio City, CA (US); Likun FEI, Alhambra, CA (US)

(73) Assignee: University of Southern California, Los Angeles, CA (US)

(21) Appl. No.: 14/157,385

(22) Filed: Jan. 16, 2014

Related U.S. Application Data
(60) Provisional application No. 61/754,448, filed on Jan. 18, 2013.

(51) Int. Cl. A61K 47/48

(52) U.S. Cl. CPC A61K 47/48246
USPC 514/1.1; 530/300; 530/333; 435/375; 435/69.1

(57) ABSTRACT

The present invention discloses a design for a molecular delivery vehicle capable of delivering a molecular payload to a target cell and its intracellular compartments. Also disclosed are highly pH-sensitive nanoconstruct that takes advantage of the requirement of cationic charge for internalization of CPPs to mask the non-specific internalization, compositions containing nanoconstruct, and methods for forming the same.

### CROSS-REFERENCE TO RELATED APPLICATIONS

This application claims the benefit of Provisional Application No. 61/754,448, filed on Jan. 18, 2013. The above priority application is hereby incorporated herein by reference.

### SEQUENCE LISTING

This application contains sequence listing.

### FIELD OF THE INVENTION

The invention pertains to the field of drug delivery. More particularly, the present invention relates to a molecular complex capable of delivering a molecular payload across cellular membrane to reach intracellular compartments in a pH-dependent manner as well as methods for forming and using the same.

### BACKGROUND OF THE INVENTION

The use of many potential biotechnological products as therapeutics—including peptides, proteins, and oligonucleotides—is limited by the fact that the amount or concentration of the product reaching the specific intracellular compartments (i.e. cytosol, nucleus) of the target cells are insufficient. Over the past decade, findings on a class of peptides, cell penetrating peptides (CPPs), have indicated that these hydrophilic oligopeptides are able to gain access to the cytosol and/or nucleus, and can shuttle bioactive macromolecules to this compartment. However, despite intensive investigation into the potential utilization of CPPs, no breakthroughs have been reported on the success of CPPs in macromolecular delivery in vivo. One major reason for the difficulty is due to the lack of selectivity for target cells and the low quantity of intracellular transport to cytosolic or nuclear sites.

Traditional targeting approaches have been attempted through the direct attachment of targeting ligands to the CPPs. However, this approach has a major drawback due to the tendency of the CPP's cationic charge to overcome the targeting efficiency of attached ligands.

Various other attempts have been made in the prior art to improve the selectivity and transport efficiency of CPPs. In one approach, it was observed that certain peptides may abolish cellular uptake of CPPs. For example, results on the evaluation of a cationic CPP, oligoarginine (R)₆, have shown that two small peptides with opposite charges, e.g., (E)₆ and (R)₆, can completely abolish the cell membrane binding and cellular uptake abilities of oligoarginine if they are covalently linked. Interestingly, when these two small peptides are mixed as separate molecules at low concentrations, they cannot form a stable complex to disturb R₆'s abilities for membrane biding and cellular update. The ability of the E/R peptide to abolish R₆'s cell penetrating property lends itself naturally as a design element for making a switchable CPP in which the ability of a CPP to penetrate a cell can be made to turn on or off by the attachment and removal of a masking group.

Prior art methods have attempted to take advantage of this design concept by directly attaching an anionic sequence via an enzyme-cleavable linkage to the R₆ CPP. However, this approach is problematic because it depends on specific enzymes to cleave the anionic sequence on site, yet these specific enzymes generally have slow cleavage efficiency and are also present in non-target sites, thereby, defeating the purpose of selectivity.

In another approach, it was recognized that the unique acidities of different cellular microenvironments may be used as an environmental trigger to selectively activate CPPs. In this approach, it was thought that the surface binding and internalization of CPPs may be triggered in a mildly acidic pH environment, such as the acidic tumor microenvironment or the acidic early endosomes of cells. To this end, prior art methods have tried to attach the masking group via acid-sensitive chemical bonds. However, this approach had only met with limited success because chemical reactivity generally cannot provide efficient hydrolysis with such a mild acidification (i.e. from pH 7.4 to 6). For example, lysyl-modification of MAP with citraconic anhydride required extremely acidic pH (pH 3-4) and long treatment times to remove the masking group.

In yet another prior art approach, polymeric systems such as pH-sensitive micelle delivery systems and pH-sensitive liposomes have shown better success in achieving systems responsive to sharp pH changes in the pH ranges between 6-7.4. However, these types of polymeric nanoparticles still face other shortcomings including serum stability, insufficient release rates of encapsulated drug, as well as the intrinsic limitation of having a larger molecular size which prevents adequate penetration in the tumor space (>50 μm) to access the acidic pH microenvironment.

Therefore, overcoming the hurdle of targeted CPP delivery remains a challenge in the field.

### SUMMARY OF THE INVENTION

In view of the above, it is one object of the present invention to provide a method of delivering a molecular payload to the surface of cells or across the cellular membrane to reach desired intracellular compartment.

It is also one object of the present invention to provide compositions and pharmaceutical formulations that are capable of efficiently distributing biologically active ingredient to the targeted cellular destinations, either at the cell surface or in intracellular compartments.

It still another object of the present invention to provide efficient and effective methods for designing, constructing, and manufacturing molecular entities that are useful as pharmaceutics or research tools for efficient delivery and targeting of said molecular entities to desired cellular destinations.

These and other objects of the present invention are satisfied by the discovery and design of highly pH-sensitive co-oligopeptides that may be fused to a CPP as a masking group to mask the cationic charge and prevent the non-specific internalization of the construct in non-target cells.

Accordingly, a first aspect of the prevent invention is directed to a pH-sensitive cellular targeting nanoconstruct useful as a cellular targeting and delivery vehicle for a molecular payload and methods for forming thereof. Nanoconstruct in accordance with this aspect of the present invention will generally include a cell-targeting element comprising a CPP operatively linked to a pH-sensitive masking element comprising an oligopeptide capable of masking the cationic charge of the CPP in a pH-dependent manner.

The cell penetrating sequence of the targeting group may be selected from any currently known or future discovered CPP sequences. In a preferred embodiment, the targeting group comprises a cationic oligopeptide sequence of about 30 amino acids in length. In a more preferred embodiment, the targeting group is less than 30 amino acids. In still another preferred embodiment, the targeting group comprises a CPP sequence selected from Table 1.

The general criteria for a CPP sequence is the inclusion of cationic arginine (R) and/or lysine (K) residues. Although not necessary, arginine residues are preferably in a clustered orientation (e.g. —RRR—) as opposed to a mixed orientation (e.g. —RXR— where X is any amino acid). For amphipathic CPPs, K residues, rather than R, are preferred.

The masking element is generally a pH-sensitive oligopeptide containing repeating units of the histidine (H)-glutamic acid (E) dipeptide having the general formula (HE)n. The length of the masking element is generally about 20 to 80 amino acids. In a preferred embodiment, the masking element is one having the general formula (HE)ₙ wherein n is between 10-40. In another preferred embodiment, n preferably matches the cationic charge and/or length of the CPP sequence to be masked. Alternatively, E residues in the co-peptide can be individually replaced with aspartic acid (D), which has anionic properties similar to E. It will be appreciated by those skilled in the art that the anionic charges of the masking element are pH-dependent. Thus, the acidic environment of the cells become a trigger to switch on/off the masking function of the masking element.

Linkage between the masking element and the targeting element may be direct linking or via a peptide linker sequence. When a linker peptide is used, the linker peptide may be cleavable (e.g. disulfide, enzyme-sensitive) or non-cleavable. Preferable linker sequences are flexible linkers to allow conformational motions of the cell targeting element and the masking element so that they may effectively interact with each other to modulate the cationic charges of the cell targeting element. Exemplary linking peptides may be a peptide having the general formula (GGGGS)ₙ wherein n is 3 to 6, or (G)ₙ wherein n is 4 to 8.

The molecular payload will generally be a bioactive agent having a desirable bioactivity to effect cellular changes, or a label (e.g. radioactive, fluorescent) to enable imaging. Exemplary bioactive payloads may include small molecules, peptides, or protein drugs, but are not limited thereto. Small molecules may be linked to any of the amino acids in the CPP-HE sequence via chemical methods. In an alternative embodiment, additional amino acids may be linked to the CPP-HE sequence to allow for conjugation. For example, small molecules and fluorescent tags can be linked to the reactive amine- or carboxylic acid groups in the N- or C-terminal of the CPP-HE sequence. In still another alternative embodiment, the CPP-HE sequence can be synthesized with one or more cysteine (C) residues to allow for thiol-linkage. Peptide or protein drugs can be conjugated via similar methods, or incorporated into the plasmid during recombinant synthesis of the CPP-HE sequence.

The above described nanoconstruct may be formed by either recombinant methods or synthetic methods. In some preferred embodiments, the nanoconstruct is produced recombinantly by constructing an expression vector comprising a sequence encoding a pH-sensitive nanoconstruct as describe above; producing the pH-sensitive nanoconstruct in a suitable expression system; and collecting the produced pH-sensitive nanoconstruct.

The expression vector, expression system and methods for collecting or purifying the resulting molecular construct may be done using any suitable vector system and purification method generally known in the art.

When a molecular payload as described above is attached to a nanoconstruct designed for a targeted cell, a cell-targeted molecular conjugate is formed. Accordingly, a second aspect of the present invention is directed to a cell-targeted molecular conjugate capable selectively targeting a targeted cell substantially as described above and methods for forming thereof.

Depending on the mode of administration and the targeted cellular environment, the cell-targeted molecular conjugate may be formulated into a pharmaceutical composition by placing it in a physiologically acceptable carrier. Accordingly, a third aspect of the present invention is directed to a pharmaceutical composition and method for formulating thereof.

In a fourth aspect, the present invention also provides a method of delivering a biologically active agent to a desired cellular destination by utilizing a pH-sensitive cell-targeting nanoconstruct as described above. Methods in accordance with this aspect of the invention generally include the step of attaching a desired biologically active agent to a suitable pH-sensitive cell targeting nanoconstruct to form a molecular conjugate; and contacting the resulting molecular conjugate with the targeted cell. The biologically active agent preferably has a predetermined activity in a target cell which, when delivered to the cell, will achieve a predetermined desired effect. Additionally, biologically active agents that may be suitably delivered will generally have a reactive site for attachment to the pH-sensitive molecular targeting construct. Criteria for the design and construction of the pH-sensitive cell targeting construct are same as described above.

The pH-sensitive nanoconstruct of the present invention has at least the following advantages: Because the membrane-penetrating properties of the targeting group are masked first by the masking group, non-specific binding to other types of cells is avoided. As a result, the nanoconstruct of the present invention will be able to target and release the biologically active agent in specific cells with the right acidic environment, thereby, selectively exerting intended biological effect at the desired cellular/nuclear site and reduces unwanted side-effects and toxicities. The ability of the nanoconstruct of the present invention to respond rapidly in mildly acidic environment also allows the construct to function effectively in mildly acidic environments such as endosomes where the acidic is only in the range of about pH 7.4 to about pH 6.5. Moreover, because the nanoconstruct of the present invention is a polypeptide, it also has the advantage of being biodegradable and low or no cytotoxicity.

Other features, objects, and advantages of the invention will be apparent from the description and the accompanying drawings, and from the appended claims.

### BRIEF DESCRIPTION OF THE DRAWINGS

FIG. 1 illustrates exemplary applications of HE-MAP nanocomplex. (A). At the surface of normal (non-tumorigenic) cells (pH 7.4), the nanocomplex will be in its inactive form, and not bind to the surface or be internalized. At the surface of tumor cells, where the pH is mildly acidic (pH 6.5-7), the nanocomplex will be activated, exposing the membrane-permeable CPP for subsequent binding and internalization. (B). At the surface of non-target cells that do not express a targeted receptor for RME, the nanocomplex will be in its inactive form and not bind to the surface or be internalized. Ligand attachment to un-masked CPPs has shown to be ineffective in biological targeting methods via RME, resulting in high internalization in non-target cells due to the high-cationic charge of CCPs. At the surface of target cells, the nanocomplex will be internalized via RME where exposure to the acidic endosomal environment will lead to activation of the membrane-permeability of the CPP.

FIG. 2 shows an exemplary SDS-PAGE with Coomassie blue staining of purified GST-HE-MAP. Crude extracts (supernatant) were obtained from bacterial cell lysate and loaded on (A) Ni-NTA or (B) GSH agarose columns. (A) The Ni-NTA agarose column was washed with buffer containing 20 mMimidazole, and GST-fusion proteins were eluted by buffer containing 250 mM imidazole. Lane 1: Molecular weight marker. Lane 2: Crude extract (supernatant). Lane 3: Ni-NTA purified fusion protein. (B) The GSH agarose column was washed with PBS, and GST-fusion proteins were eluted with PBS containing 50 mM GSH. Lane 1: Molecular weight marker. Lane 2: GSH purified fusion protein.

FIG. 3 shows the surface binding (A) and internalization (B) of ¹²⁵I-GST-HE-MAP in HeLa cells at various pH. HeLa cells were treated with 5 μg/mL ¹²⁵I-GST-HE-MAP fusion protein for 1 h at 37° C. at the indicated pH. The cell monolayers were washed with PBS, and detached with trypsin-EDTA. The cells were centrifuged to separate the trypsin removable supernatant (i.e. surface bound) from the cell pellet. The cell pellet was washed with ice-cold PBS, and the amount of ¹²⁵I-GST-HE-MAP per cell monolayer was determined after measuring the radioactivity.

FIG. 4 shows images of confocal analysis of GST-HE-MAP internalization. Cells were treated with 10 μg/mL of RITC-labeled GST-HE-MAP for 1 h at 37° C. at a pH of (A) 7.4 or (B) 6.5 and analyzed by confocal microscopy at Ex 372/Em 456 (blue DAPI stained nuclei) and Ex 570 nm/Em 595 nm (red punctate RITC-GST-HE-MAP staining)

FIG. 5 shows (A) SDS-PAGE and Commassie blue staining of GST=agarose purified GST-HE-MAP fusion protein. Lane 1: molecular weight marker. Lane 2: GST-HE-MAP (expected molecular weight=31.8 kDa). (B, C) HeLa cells were treated with 3 g/mL ¹²⁵I-GST-HE-MAP fusion protein for 1 h at 37° C. and the indicated pH. The cell monolayers were washed with PBS, and detached with trypsin-EDTA. The cells were centrifuged to separate the (B) trypsin removable supernatant (i.e. surface bound) and the (C) cell pellet (i.e. internalized)¹²⁵I-GST-HE-MAP.

FIG. 6 shows Comparison of a cationic CPP (TAT) and amphipathic CPP (MAP). HeLa cells were treated with 3 g/mL ¹²⁵I-labeled (A) MAP fusion proteins or (B) TAT fusion proteins for 1 h at 37° C. and the indicated pH. The cell monolayers were washed with PBS, and detached with trypsin-EDTA, and the amount in the cell pellet (i.e. internalized) was measured.

### DETAILED DESCRIPTION

Unless otherwise defined, all technical and scientific terms used herein have the same meaning as commonly understood by one of ordinary skill in the art to which this invention pertains. In case of conflict, the present document, including definitions, will control. All publications, patent applications, patents and other references mentioned herein are incorporated by reference in their entirety. The materials, methods, and examples disclosed herein are illustrative only and not intended to be limiting.

### DEFINITIONS

As used herein, the term “CPP” refers to a class of peptides known as Cell Penetrating Peptides. They are a group of small cationic or amphipathic peptides that can be transported across plasma membrane to reach intracellular compartments such as the cytosol and nucleus. A review of these peptides can be found in Patel et al. Cell Penetrating Peptides: Intracellular pathways and Pharmaceutical Perspectives, Pharm. Res. 24: 1977-1992 (2007) (the entire content of which is incorporated herein by reference).

As used herein, the term “MAP” refers to the Model Amphipathic Peptide, the sequence of which is shown in Table 1.

As used herein, the term “peptide” is a sequence of 2 to 25 amino acids (e.g. as defined hereinabove) or peptidic residues having one or more open valences. The sequence may be linear or cyclic. For example, a cyclic peptide can be prepared or may result from the formation of disulfide bridges between two cysteine residues in a sequence. A peptide can be linked through the carboxy terminus, the amino terminus, or through any other convenient point of attachment, such as, for example, through the sulfur of a cysteine. Peptide derivatives can be prepared as disclosed in U.S. Pat. Nos. 4,612,302; 4,853,371; and 4,684,620. Peptide sequences specifically recited herein are written with the amino terminus on the left and the carboxy terminus on the right.

The term “polypeptide” refers to a biopolymer compound made up of a single chain of amino acid residues linked by peptide bonds. The term “protein” as used herein may be synonymous with the term “polypeptide” or may refer, in addition, to a complex of two or more polypeptides.

The terms “residue” or “amino acid residue” or “amino acid” are used interchangeably herein to refer to an amino acid that is incorporated into a protein, polypeptide, or peptide (collectively, “protein”). The amino acid may be a naturally occurring amino acid and, unless otherwise limited, may encompass known analogues of natural amino acids that can function in a similar manner as naturally occurring amino acids.

The term “substantially the same” refers to nucleic acid or amino acid sequences having sequence variation that do not materially affect the nature of the protein (i.e. the structure, stability characteristics, substrate specificity and/or biological activity of the protein). With particular reference to nucleic acid sequences, the term “substantially the same” is intended to refer to the coding region and to conserved sequences governing expression, and refers primarily to degenerate codons encoding the same amino acid, or alternate codons encoding conservative substitute amino acids in the encoded polypeptide. With reference to amino acid sequences, the term “substantially the same” refers generally to conservative substitutions and/or variations in regions of the polypeptide not involved in determination of structure or function.

As used herein, “expression” includes the process by which polynucleotides are transcribed into mRNA and translated into peptides, polypeptides, or proteins. If the polynucleotide is derived from genomic DNA, expression may include splicing of the mRNA, if an appropriate eukaryotic host is selected. Regulatory elements required for expression include promoter sequences to bind RNA polymerase and transcription initiation sequences for ribosome binding. For example, a bacterial expression vector includes a promoter such as the lac promoter and for transcription initiation the Shine-Dalgamo sequence and the start codon AUG (Sambrook, J., Fritsh, E. F., and Maniatis, T. Molecular Cloning: A Laboratory Manual. 2nd, ed., Cold Spring Harbor Laboratory, Cold Spring Harbor Laboratory Press, Cold Spring Harbor, N.Y., 1989). Similarly, a eukaryotic expression vector includes a heterologous or homologous promoter for RNA polymerase II, a downstream polyadenylation signal, the start codon AUG, and a termination codon for detachment of the ribosome. Such vectors can be obtained commercially or assembled by the sequences described in methods well known in the art, for example, the methods described below for constructing vectors in general.

The term “vector” refers to a nucleic acid construct designed for transfer between different host cells. An “expression vector” refers to a vector that has the ability to incorporate and express heterologous DNA fragments in a foreign cell. Many prokaryotic and eukaryotic expression vectors are commercially available. Selection of appropriate expression vectors is within the knowledge of those having skill in the art. Accordingly, an “expression cassette” or “expression vector” is a nucleic acid construct generated recombinantly or synthetically, with a series of specified nucleic acid elements that permit transcription of a particular nucleic acid in a target cell. The recombinant expression cassette can be incorporated into a plasmid, chromosome, mitochondrial DNA, plastid DNA, virus, or nucleic acid fragment. Typically, the recombinant expression cassette portion of an expression vector includes, among other sequences, a nucleic acid sequence to be transcribed and a promoter.

Generally, “operably linked” means that the DNA and/or peptide sequences being linked are contiguous, and, in the case of a secretory leader, contiguous and in reading frame. However, “operably linked” elements, e.g., enhancers, do not have to be contiguous. For example, two segments of polypeptides may be operatively linked via a direct polypeptide bond or be linked via a linker molecule.

The term “small molecule” refers to compounds that are not macromolecules (see, e.g., Karp (2000) Bioinformatics Ontology 16:269-85; Verkman (2004) AJP-Cell Physiol. 286:465-74). Thus, small molecules are often considered those compounds that are, e.g., less than one thousand daltons (e.g., Voet and Voet, Biochemistry, 2.sup.nd ed., ed. N. Rose, Wiley and Sons, New York, 14 (1995)). For example, Davis et al. (2005) Proc. Natl. Acad. Sci. USA 102:5981-86, use the phrase small molecule to indicate folates, methotrexate, and neuropeptides, while Halpin and Harbury (2004) PLos Biology 2:1022-30, use the phrase to indicate small molecule gene products, e.g., DNAs, RNAs and peptides. Examples of natural small molecules include, but are not limited to, cholesterols, neurotransmitters, aptamers and siRNAs; synthesized small molecules include, but are not limited to, various chemicals listed in numerous commercially available small molecule databases, e.g., FCD (Fine Chemicals Database), SMID (Small Molecule Interaction Database), ChEBI (Chemical Entities of Biological Interest), and CSD (Cambridge Structural Database) (see, e.g., Alfarano et al. (2005) Nuc. Acids Res. Database Issue 33:D416-24).

The term “Carriers” as used herein include pharmaceutically acceptable carriers, excipients, or stabilizers which are nontoxic to the cell or mammal being exposed thereto at the dosages and concentrations employed. Often the physiologically acceptable carrier is an aqueous pH buffered solution. Examples of physiologically acceptable carriers include buffers such as phosphate, citrate, and other organic acids; antioxidants including ascorbic acid; low molecular weight (less than about 10 residues) polypeptide; proteins, such as serum albumin, gelatin, or immunoglobulins; hydrophilic polymers such as polyvinylpyrrolidone; amino acids such as glycine, glutamine, asparagine, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, polyethylene glycol (PEG), and PLURONICS™.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention. A “pharmaceutically acceptable counterion” is an ionic portion of a salt that is not toxic when released from the salt upon administration to a recipient.

Pharmaceutically acceptable salts may be obtained using standard procedures well known in the art, for example by reacting a sufficiently basic compound such as an amine with a suitable acid affording a physiologically acceptable anion. Alkali metal (e.g., sodium, potassium or lithium) or alkaline earth metal (e.g., calcium) salts of carboxylic acids can also be made.

Design Principle of pH-Sensitive Nanoconstructs

It is an unexpected discovery of the present invention that a molecular group having an anionic charge that is sensitive to pH may be coupled to a CPP to act as a masking element. Such coupling allows one to take advantage of the fact that cationic charges are required for internalization of CPPs. By utilizing the differences in the acidic environments of different cells, one may couple different masking groups to achieve cell selectivity, thereby, overcoming all the problems of prior art attempts in utilizing CPPs as delivery vehicles.

It is noted that CPP sequences used in the present invention are generally short sequence of amino acids containing 30 amino acids or less. Numerous CPP sequences are known in the art. They are generally classified as either cationic (cCPP) containing arginine and lysine residues, or amphipathic (aCPP) containing lysine residues mixed with hydrophobic amino acids such as leucine and tryptophan. Table 1 shows some exemplary CPPs.

To further illustrate the present invention, described in detail below is an exemplary embodiment utilizing a highly pH-sensitive cooligopeptide sequence fused to a CPP to mask the cationic charge and prevent the non-specific internalization of the construct in non-target cells.

Conceivably, the masking sequence can be applied to any CPP sequence that depends on lysine and/or arginine residues for efficient internalization. The pH-sensitive co-oligopeptide sequence consists of histidine-glutamic acid (HE) repeats. Incorporation of histidine residues in CPP sequences has been shown to facilitate proton influx in endosomes for endosomolytic activity and increase lipophilicity of the CPP sequence, and histidine-CPP analogs show higher internalization at pH 6 than pH 7.4. In the following example, a histidine is mixed with glutamic acid for use as a pH-sensitive blocking sequence to mask the cationic charge of the CPP sequence. The imidazole group of histidine (pKa≈6) in the HE co-oligopeptide sequence is neutral at physiological pH 7.4, allowing for the electrostatic interaction between anionic γ-carboxylate groups of glutamic acid (pKa≈4) and cationic ε-amino groups of lysine (pKa≈10) or guanidino groups of arginine (pKa≈12). The length of the (HE) repeats can be easily customizable to accommodate the number of cationic charges in the CPP sequence.

The masking group and the targeting group may be linked via reducible or non-reducible peptide sequences. The construct may be made via several different methods including (a) recombinant production in bacteria (for example, in E. coli) followed by purification using analytical methods such as affinity chromatography (i.e. Ni-NTA column chromatography which recognizes the H in the sequence) or size exclusion chromatography; (b) recombinant production in mammalian cells followed by purification using analytical methods such as affinity chromatography or size exclusion chromatography; or (c) solid phase synthesis.

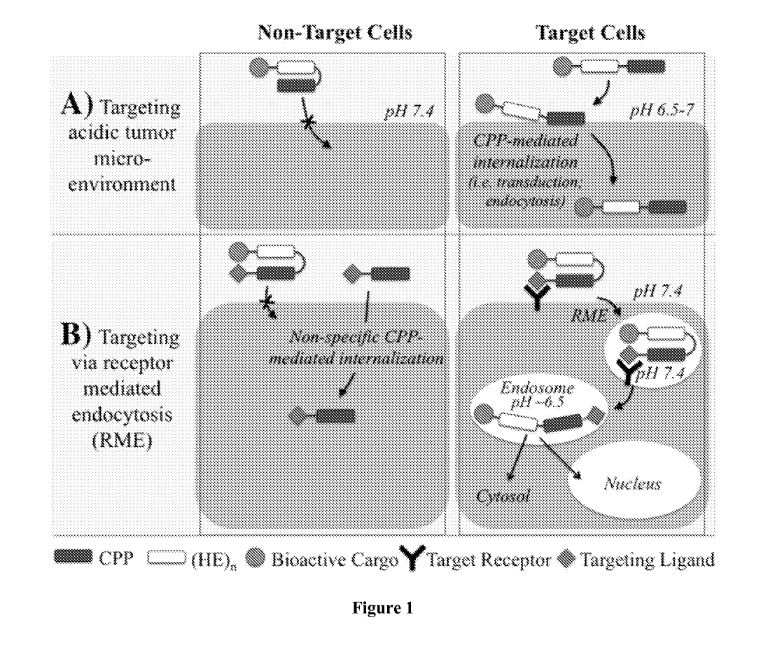

While not intending to be bound by any particular theory, it is believed that after exposure to mildly acidic pH, the imidazole group in histidine will undergo a change from neutral to cationic, thus neutralizing the charge of the HE-peptide and freeing the electrostatic interaction of the glutamic acid residues with cationic residues of the CPP. Therefore, the membrane activity of the CPP will be rapidly regenerated at mildly-acidic pH. This regeneration process is similar to the rapid dissociation between ligands and receptors inside acidic endosomes triggered by the change of protein conformation. The hypothetical model and potential applications of this nanocomplex in drug delivery are shown in FIG. 1.

### Demonstrative Example 1

In this demonstrative example, an exemplary nanoconstructs were produced using the amphipathic CPP, MAP (KLALKLALKALKAALKLA) (SEQ ID No. 8), which is internalized avidly via endocytosis, and preliminary studies in our laboratory have shown that it has linker-dependent cargo delivery capabilities to the cytosolic or nuclear compartment. In addition to its high intracellular accumulation, MAP is also one of the most stable CPPs with a long pharmacokinetic half-life of greater than 72 h. The HE-MAP nanoconstruct was produced as a fusion protein with glutathione S-transferase (GST), which served as an aid for purification and as a cargo protein to study the pH-sensitive HE-MAP-mediated delivery.

### Experimental Methods

### Production of Nanocomplex

The nanocomplex was prepared as a fusion protein between GST, a 10-mer HE-oligopeptide sequence ((HE)₁₀), a short pentaglycine (G₅) linker, and MAP (“GST-HE-MAP”). DNA sequence encoding the GST-HE-MAP fusion protein was cloned in the Escherichia coli (E. coli). The two ssDNAs were synthesized by ValueGene (San Diego, Calif.) and were annealed together followed by PCR amplification to form the intact dsDNA. The dsDNAs were then inserted into pGEX-4T-1 vectors (GE Healthcare Life Sciences, Piscataway, N.J.) through BamHI and NotI cleavage sites. After the ligation, the plasmids were transformed into E. coli (DH5α strain). The positive clones were selected by colony PCR and are sent for sequencing (GeneWiz, San Diego, Calif.). The plasmids with correct insertions were transformed into E. coli expression strain BL21. For expression of recombinant protein, the bacteria carrying the plasmids were incubated in lysogeny broth (LB) media with 50 μg/mL ampicillin at 37° C., 250 rpm until the A600 of media reached 0.6-0.8. Isopropyl β-D-1-thiogalactopyranoside was added into LB media to a final concentration of 0.2 mM. After 4 h of additional incubation, the bacteria were collected and stored at −80° C. Expression of the GST-fusion protein was monitored by SDS-PAGE followed by Coomassie blue staining

### Purification and Labeling of GST-HE-MAP

The recombinant GST-fusion protein was purified from crude extracts using glutathione (GSH) (which recognizes GST) or nickel nitriloacetic acid (Ni-NTA) (which recognizes histidine) agarose beads (Qiagen, Valencia, Calif.). The bacteria were resuspended in phosphate buffered saline (PBS, for GST agarose) or lysis buffer (50 mM NaH2PO4, 300 mM NaCl, and 10 mM imidazole, pH 8.0 for Ni-NTA agarose). After the bacteria were lysed by sonification, crude extracts (supernatant) were obtained from lysate by centrifugation. The crude extracts were loaded on GSH or Ni-NTA agarose columns prebalanced with either buffer mentioned above. The columns were then washed with PBS (for GSH agarose) or wash buffer (50 mM NaH2PO4, 300 mM NaCl, and 20 mM imidazole, pH 8.0 for Ni-NTA agarose). Finally, GST-fusion protein was eluted with PBS containing 50 mM GSH (for GSH agarose) or with elution buffer (50 mM NaH₂PO₄, 300 mM NaCl, and 250 mM imidazole, pH 8.0 for Ni-NTA agarose). The excess GSH or imidazole was removed by dialysis against PBS (regenerated cellulose, 12,000-14,000 MW cutoff, Spectrum Laboratories, Inc., Rancho Dominguez, Calif.). During purification, GST-fusion proteins were monitored by UV absorbance at a wavelength of 280 nm, and SDS-PAGE with Coomassie blue staining. The band densities were measured using Quantity One software (BioRad, Hercules, Calif.) and used to determine fusion protein purity.

The purified GST-fusion proteins were radiolabeled with either Na¹²⁵I or rhodamine isothiocyanate (RITC, Sigma, St. Louis, Mo.). Radioiodination was performed using the chloramine T method as previously described elsewhere, and ¹²⁵I-GST-HE-MAP was purified by size exclusion chromatography using a Sephadex G50 (GE Healthcare Life Sciences). RITC-labeling was performed by incubating RITC with GST-HE-MAP at a 1.5-fold molar excess overnight at 4° C., and RITCGST-HE-MAP was purified by size exclusion chromatography (G50 Sephadex, GE healthcare, PBS mobile phase).

### Cell Culture Assay

HeLa cell monolayers were cultured at 37° C., 5% CO₂ in RPMI 1640 (Mediatech, Manassas, Va.) supplemented with 10% fetal bovine serum. For quantitative internalization assays, confluent cell monolayers grown in 6-well culture plates were treated with RPMI 1640 media at pH 6.0, 6.3, 6.6, 6.9, 7.2, or 7.5 containing 5 μg/mL ¹²⁵IGST-HE-MAP fusion protein for 1 h at 37° C. The cell monolayers were washed with cold PBS, detached with trypsin-EDTA, and centrifuged to separate the trypsin removable supernatant (i.e. surface bound) and the cell pellet (i.e. internalized)¹²⁵I-GST-HE-MAP. The samples were analyzed for radioactivity using a Gamma counter (Packard, Downers Grove, Ill.). For qualitative confocal microscopy assays, cells were grown overnight on coverslips in 12-well culture plates. Cell monolayers were treated with RPMI 1640 media at pH 6.5 or 7.4 containing 10 μg/mL RITC-GST-HE-MAP fusion protein for 1 h at 37° C. The cell monolayers were washed with 0.5 mg/mL heparin and PBS to remove surface bound protein, and fixed in 4% paraformaldehyde for 15 min. Afterward, cells were washed with PBS, and nuclei were stained with 300 nM 4′,6-diamidino-2-phenylindole (DAPI) (Sigma) for 15 min. Cells were washed twice more with PBS. The cover slips were mounted on micro slides with anti-fade reagent (Invitrogen, Eugene, Oreg.). Pictures were obtained using confocal laser scanning microscope (CLSM) (Zeiss LSM 510 Meta, Carl Zeiss, Jena, Germany).

### Discovery and Interpretation

### Production and Purification

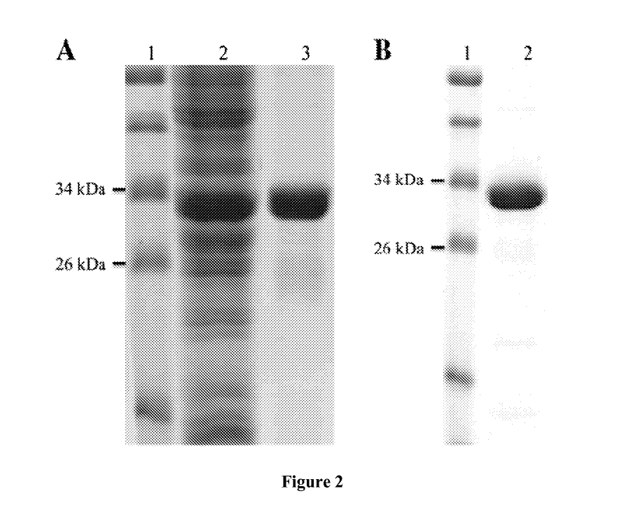

A peptide sequence containing (HE)₁₀ and MAP linked via a short (G)₅ peptide linker was fused to GST for recombinant production in E. coli. The recombinant fusion protein, GST-HE-MAP, was purified from crude extracts with either Ni-NTA or GSH agarose resins. First, the use of Ni-NTA agarose to purify the fusion protein was evaluated. Since the oligohistidine in GST-HE-MAP was mixed with anionic glutamic acid residues in the sequence, it was not clear whether or not it could be recognized by the Ni-NTA resin. As shown in FIG. 2A, GST-HE-MAP could be purified using the histidine residues as a recognition site, resulting in ˜92% purity as determined by SDS-PAGE with Coomassie blue staining Therefore, the incorporation of oligohistidine in the sequence for the nanoconstruct adds an additional advantage in the purification methods following recombinant production. Alternatively, the fusion protein was also purified from crude extracts using GSH agarose chromatography, resulting in ˜94% purity as determined by SDS-PAGE with Coomassie blue staining (FIG. 2B).

### Quantitative Analysis of Binding and Internalization of ¹²⁵ I-GST-HEMAP at Various pH

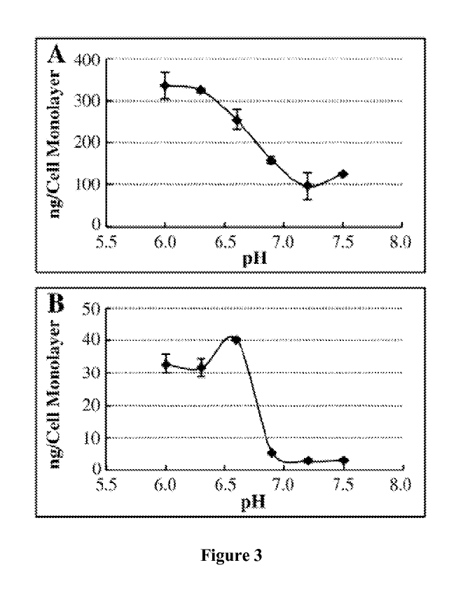

The binding and internalization of ¹²⁵I-GST-HE-MAP fusion protein were determined at various pH values, and compared to the net charge of the HE-MAP sequence at the respective pH. As shown in Table 2, the net charge of the sequence is cationic below its isoelectric point (pI=6.5). As shown in FIGS. 3A and B, the ¹²⁵I-GST-HE-MAP fusion protein exhibited a pH-dependent surface binding and internalization profiles that correlated with the calculated net charge of the HE-MAP sequence. The surface binding of ¹²⁵I-GST-HE-MAP decreased with increasing pH, and leveled off at pH 6.3 and below (FIG. 3A), which correlates well with the net charge (Table 2). Similarly, the internalization of the fusion protein also decreased with increasing pH, with an inflection point near the pI of the HE-MAP construct (FIG. 3B). The internalization was very low at pH levels above the pI, where the charge of the complex is anionic, while the internalization at pH 6.6 is ˜14-fold higher than at pH 7.2 and above. There is a drop in internalization at pH levels below 6.3, which is most likely due to inhibition of endocytosis in this pH range, however, the internalization is still ˜11-fold higher at pH 6-6.3 than at pH 7.2 and above. Taken together, these data strongly indicate that the HE-MAP construct exhibits pH-dependent binding and internalization at the expected pH values of the mildly acidic tumor microenvironment or early endosome.

### Qualitative Analysis of RITC-GST-HE-MAP at Acidic and Neutral pH

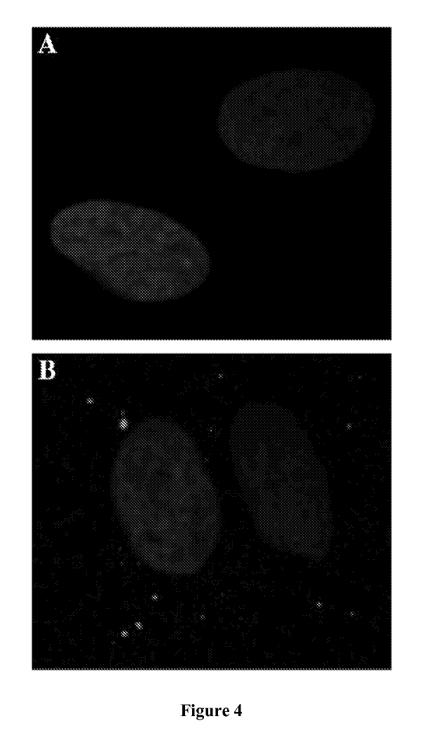

In order to verify the quantitative comparison of internalization of GST-HE-MAP at various pH, the internalization of RITC-labeled fusion protein was determined using confocal microscopy. As shown in FIG. 4, there was no observable red-fluorescent staining for cells incubated with the fusion protein at pH 7.4. On the other hand, cells incubated with the fusion protein at pH 6.5 showed punctate vesicular staining throughout the cell, which is consistent with the localization of MAP and its protein cargo conjugates. These data confirm the quantitative analysis results and show that the GST-HE-MAP conjugate demonstrates pH-sensitive internalization.

### Conclusion

The nanoconstructs disclosed herein can be utilized in several different areas including the exploitation of the acidic tumor microenvironment in diagnosis and targeting of tumors, and in application of CPPs in targeted delivery of macromolecular drugs. Depending on the type of CPP utilized, these constructs will be immensely useful as carriers of macromolecular drugs to either the cytosolic and/or nuclear compartments of target cells. These nanoconstructs are also easily amenable to attachment of other molecules, including PEGylation to improve pharmacokinetic and/or biodistribution properties. Since the pH-sensitivity of the nanocomplex depends on the charge interaction between HE and the cationic CPP, the physical chemical properties of the cargo or other additional molecules will need to be considered. This novel design of a CPP carrier has the potential to not only provide a new method in targeting of bioactive drugs, but could also lead to advancement of the field of biotechnology-derived drugs (i.e. proteins and peptides) as therapeutics.

### Demonstrative Example 2

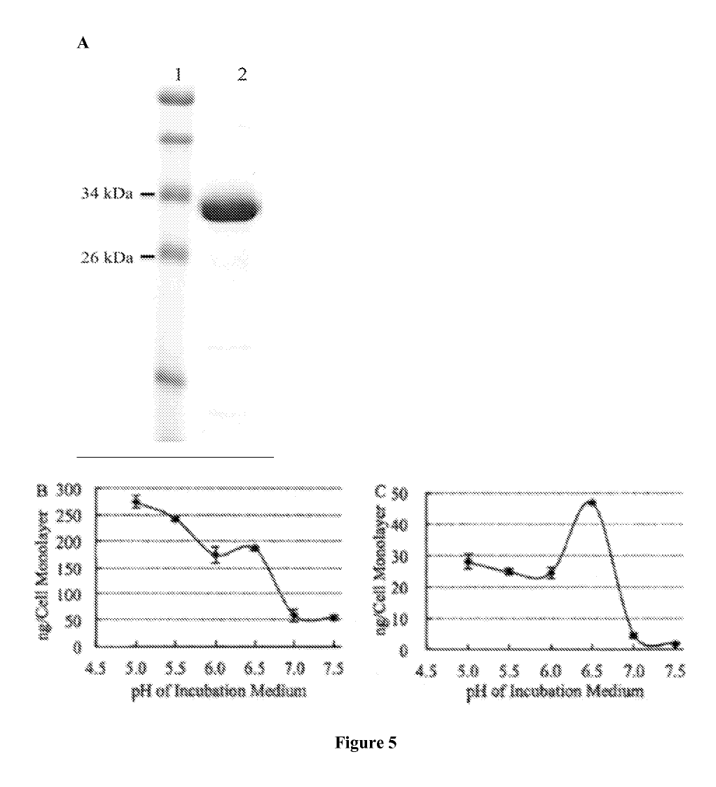

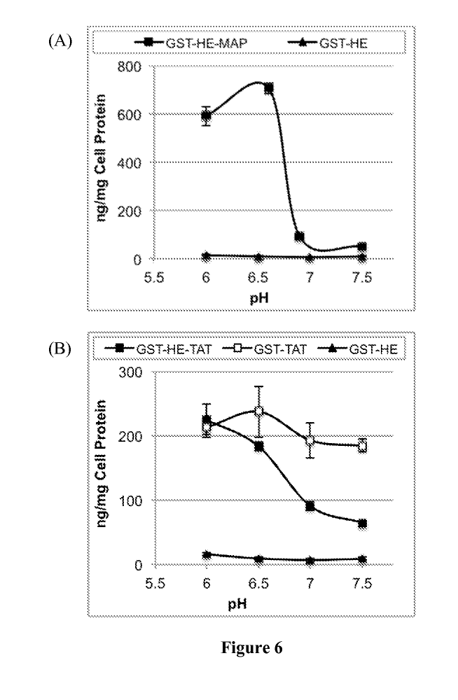

Another exemplary pH-sensitive nanoconstruct was made by recombinantly producing a fusion protein comprising a peptide containing (HE)₁₀ and a CPP (TAT), and compared to HE-MAP. As shown in FIG. 6A and our recent publication, the GST-HE-MAP fusion protein exhibited a pH-dependent internalization profile, where the internalization at mildly acidic pH (6.6 and below) was ˜10-fold higher than at pH 6.9 and above. GST-HE-TAT, GST-TAT, and GST-HE were produced in E. coli and purified by GST affinity chromatography using similar methodology as described in FIG. 5. As shown in FIG. 6B, the internalization of GST-HE-TAT also exhibited pH sensitivity between pH 6.5 and 7, while GST-TAT and GST-HE were not pH-sensitive. The internalization of GST-HE-TAT was similar to GST-TAT in mildly acidic pH, and reduced by ˜4-fold at pH 7.5 (FIG. 6B). Furthermore, the internalization of GST-HE (FIGS. 6A and B) was very low across the entire pH range, indicating that increase of internalization of GST-HE-MAP and GST-HE-TAT in the mildly acidic pH was due to the CPP-mediated uptake and not due to an increased non-specific internalization at the low pH.

### Targeted Delivery of Bioactive Agents to Desired Cellular Destinations

See the GST-examples above.

Compositions Containing pH-Dependent Target Selective Agents

The nanoconstructs and nanoconstruct-bioactive agents would be sterilized by passing through a 0.22 μm filter and stored in phosphate buffer at 4° C. at pH approximately 7. Alternatively, the nanoconstructs and nanoconstruct-bioactive agents may be lyophilized following purification and sterilization, and stored in a desiccator at −20° C. Lyophilized product would be reconstituted in sterile deionized water prior to use. Nanoconstructs and bioactive-agents will be administered by either subcutaneous or intravenous injection.

Although the present invention has been described in terms of specific exemplary embodiments and examples, it will be appreciated that the embodiments disclosed herein are for illustrative purposes only and various modifications and alterations might be made by those skilled in the art without departing from the spirit and scope of the invention as set forth in the following claims.

| | | Classifi- |
|---|---|---|
| Peptide | Sequence | cation |
| Tat | YGRKKRRQRRR | Cationic |
| (47-57) | (SEQ ID 1) | (PD) |
| (HIV-1) | | |
| Oligo- | Rₙ/Kₙ (n = 6-9) | Cationic |
| arginine/ | | (S) |
| Oligolysine | | |
| Rev | TRQARRNRRRRWRERQRGC | Cationic |
| (34-50) | (SEQ ID 2) | (PD) |

(HIV-1)

| | | |
|---|---|---|
| pVEC | LLILRRRIRKQAHAHSK | Amphipathic |
| | (SEQ ID 3) | (PD) |
| Penetratin | RQIKIWFQNRRMKWKK | Amphipathic |
| | (SEQ ID 4) | (PD) |
| M918 | MVTVLFRRLRIRRACGPPRVRV | Amphipathic |
| | (SEQ ID 5) | (PD) |
| Pep-1 | KETWWETWWTEWSQPKKKRKV | Amphipathic |
| | (SEQ ID 6) | (C) |
| Transportan | GWTKNSAGYLLGKINLKALAAL | Amphipathic |
| | AKKIL (SEQ ID 7) | (C) |
| MAP | KLALKLALKALKAALKLA | Amphipathic |
| | (SEQ ID 8) | (S) |

TABLE 2
Net charge of HE-MAP sequence.

| pH | Net charge^{a} |
|---|---|
| 7.5 | −4.3 |
| 7.2 | −3.5 |
| 6.9 | −2.2 |
| 6.6 | −0.6 |
| 6.3 | +1.2 |
| 6.0 | +2.8 |

^{a}The net charge of the HE-MAP sequence ((HE)₁₀G₅ KLALKLALKALKAALKLA) was calculated based on the individual pKa values of the ionizable groups (H pKa = 6.0; K pKa = 10.6; E pKa = 4.25; N-terminal amine pKa = 9.2; C-terminal carboxyl pKa = 2.34).

### REFERENCES

The following references are cited herein. The entire disclosure of each reference is relied upon and incorporated by reference herein.
1. Zaro J L, Shen W C. Quantitative comparison of membrane transduction and endocytosis of oligopeptides. Biochemical and Biophysical Research Communications. 2003; 307(2):241-7.
2. Zaro I L, Shen W C. Evidence that membrane transduction of oJigoargitline does not require vesicle formation. Experimental Cell Research; 2005; 307(1): 164-73.
3. Zaro J. Vekich J, Tran T. Shen W. Nuclear Localization of cell-Penetrating peptides Is Dependent on Endocytosis Rather Than Cytosolic Delivery in CHO Cells. Molecular Pharmaceutics. 2009.
4. Morris M C, Deshayes S, Heitz F. Divita O. Cell-Penetrating peptides: from molecular mechanisms to therapeutics. Biology of the Cell 2008; 100(4):201-17.
5. Pooga M, HaUbrink M. Zorko M, Langel U. Cell penetration by transportan. Faseb Journal. 1998; 12(1):67-77.
6. Elmquist A, Lindgren M, Bartfai T, Langel U. VE-cadherin-derived cell-penetrating peptide, pVEC, with carrier functions. Experimental Cell Research. 2001; 269(2):237-44.
7. Fischer R, Waizenegger T, Kohler K, Brock R. A quantitative validation of fluorophore-labelled cell-permeable peptide conjugates: fluorophore and cargo dependence of import. Biochimica Et Biophysica Acta-Biomembranes. 2002; 1564(2):365-74.
8. Zaro J L, Fei L K, Ren L, Shen W C. The influence of net charge and charge distribution on cellular uptake and cytosolic localization of arginine-rich peptides. Journal of Drug Targeting. 2011; 19(8):675-80.
9. Patel L N, Zaro J L, Shen W C. Cell penetrating peptides: Intracellular pathways and pharmaceutical perspectives. Pharmaceutical Research. 2007; 24:1977-92.
10. Vives E, Present and future of cell-penetrating peptide mediated delivery system. “Is the Trojan horse too wild to go only to Troy?” Journal of Controlled Release. 2005; 109(1-3):77-85.
11. Wotf Y, PrItt S, Ahes S. Bienert M, Lebleu B, Oehlke J. Structural requirements for cellular uptake and antisense activity of peptide nucleic acids conjugated with various peptides. Biochemistry. 2006; 45(50): 1494454.
12. Davoust J, Gruenberg J, Howell K E. 2 Threshold Valus of Low Ph Block Endocytosis at Different Stages. Embo J. 1987; 6(12):3601-9.
13. 13. Shen W C, Ryser H J P. Cis-aconityl spacer between daunomycin and macromolecular carriers—a model of pH-sensitive linkage releasing drug from a lysosomotropic conjugate. Biochemical and Biophysical Research Communications. 1981; 102(3): 1048-54.
14. Blattler W A, Kuenzi B S, Lambert J M, Senter P D. New Heterobifunctional Protein Cross-Linking Reagent that Forms an Acid-Labile Link. Biochemistry. 1985; 24:1517-24.
15. Dbawan A, Sharma V. Toxicity assessment of nanomaterials: methods and challenges. Analytical and Bioanalytical Chemistry. 2010; 398(2):589-605.
16. Shen W C. Acid-sensitive dissociation between poly(lysine) and histamine-modified poly(glutamate) as a model for drug-releasing from carriers in endosomes. Biochimica et Biopbysica Acta. 1990; 1034(1):122-4.

What is claimed is:

1. A peptide nanoconstruct for targeted cell delivery, comprising:
a cell targeting element comprising a cellular targeting peptide (CPP) having a cationic charge; and
a pH-sensitive masking element comprising a pH-sensitive oligopeptide for masking the cationic charge of the cell targeting element,
wherein the targeting element is operatively linked to the pH-sensitive masking element either directly or via an optional peptide linker.

2. The peptide nanoconstruct of claim 1, wherein said pH-sensitive oligopeptide comprises a histidine-glutamic acid repeat (HE)ₙ, wherein n=10 to 40, and wherein said glutamic acid can be individually replaced by aspartic acid (D).

3. The peptide nanoconstruct of claim 1, wherein said CPP is one comprising arginine residues in clusters of three (—RRR—) or more.

4. The peptide nanoconstruct of claim 1, wherein said CPP is amphipathic.

5. The peptide nanoconstruct of claim 1, wherein said cell targeting element further comprises a cysteine residue (C) for thiol-linkage of a molecular payload.

6. The peptide nanoconstruct of claim 1, wherein the pH-sensitive oligopeptide and the CPP have matching opposite charges.

7. The peptide nanoconstruct of claim 1, wherein the pH-sensitive oligopeptide and the CPP have the same length.

8. The peptide nanoconstruct of claim 1, wherein the linkage between the cell targeting element and the pH-sensitive masking element is via direct linkage.

9. The peptide nanoconstruct of claim 1, wherein said peptide linker is cleavable by an enzyme.

10. The peptide nanoconstruct of claim 1, wherein said peptide linker is a flexible linker comprising a peptide sequence having the general formula (GGGGS)ₙ, wherein n=3 to 6 or (G)ₙ, wherein n=4 to 8.

11. A cell-targeted molecular conjugate, comprising:
a peptide nanoconstruct according to claim 1 attached to a molecular payload with a predetermined biological activity in a target cell.

12. The molecular conjugate of claim 10, wherein the molecular payload is selected from the group consisting of a fluorescent tag, a small molecule drug, a peptide drug, and a protein drug.

13. The molecular conjugate of claim 10, wherein the molecular payload is attached to the peptide nanoconstruct via a reactive amine- or carboxylic acid group in the nanoconstruct.

14. A pharmaceutical composition, comprising:
a cell-targeted molecular conjugate of claim 10 wherein said molecular payload; and
a physiologically acceptable carrier.

15. A method of forming a peptide nanoconstruct for targeted cell delivery, comprising:
attaching a pH-sensitive masking element to a cell targeting element directly or via a peptide linker,
wherein:
said pH-sensitive masking element is a peptide comprising a histidine-glutamic acid repeat sequence (HE)ₙ with n=10 to 40 and the glutamic acid E individually replaceable with aspartic acid D,
said cell targeting element is a CPP having a cationic charge.

16. The method of claim 14, wherein attachment is accomplished by chemical attachment.

17. The method of claim 14, wherein attachment is accomplished by recombinant expression of a vector encoding the peptide sequences of the masking element and the cell targeting element in tandem.

18. A method of forming a cell-targeted molecular conjugate, comprising:
attaching a molecular payload having a predetermined biological activity in a target cell to a peptide nanoconstruct,
wherein said peptide nanoconstruct comprising a cell targeting element operatively linked to a pH-sensitive masking element directly or via a peptide linker, and wherein:
said cell targeting element comprises a cationic CPP,
said masking element comprises a pH-sensitive oligopeptide containing a histidine-glutamic acid repeat (HE)ₙ, n=10 to 40, and the glutamic acid individually replaceable with aspartic acid (D).

19. The method of claim 17, wherein said peptide linker comprises a peptide having the general formula (GGGGS)ₙ, n=3 to 6 or (G)ₙ, n=4 to 8

20. A method of delivering a biologically active agent to a targeted cell having a targeted acidic environment, comprising:
attaching the biologically active agent to a peptide nanoconstruct to form a cell-targeted molecular agent; and
contacting the cell-targeted molecular agent to a targeted cell so as to allow the cell-targeted molecular agent be internalized, thereby, delivering the biologically active agent,
wherein said peptide nanoconstruct comprises a cell-targeting element operatively linked to a pH-sensitive masking element either directly or via a peptide linker, and wherein:
said cell-targeting element comprises a cationic CPP,
said pH-sensitive masking element comprises a pH-sensitive oligopeptide containing a histidine-glutamic acid repeat (HE)ₙ, n=10-40, said glutamic acid is individually replaceable by aspartic acid D, and
said peptide linker containing a peptide repeat having the general formula (GGGGS)ₙ, n=3-6 or (G)ₙ, n=4 to 8.

21. The method of claim 19, wherein said biologically active agent is one selected from the group consisting of a fluorescent tag, a small molecule drug, a peptide drug and a protein drug.


SEQUENCE LISTING (8 sequences)

SEQ ID NO: 1 - protein, 11 aa, Artificial Sequence
  Fragment of HIV Tat (47 - 57)
YGRKKRRQRRR

SEQ ID NO: 2 - protein, 19 aa, Artificial Sequence
  Fragment of HIV-1 Rev Protein
TRQARRNRRRRWRERQRGC

SEQ ID NO: 3 - protein, 17 aa, Artificial Sequence
  A cell penetrating peptide
LLILRRRIRKQAHAHSK

SEQ ID NO: 4 - protein, 16 aa, Artificial Sequence
  A cell penetrating peptide
RQIKIWFQNRRMKWKK

SEQ ID NO: 5 - protein, 22 aa, Artificial Sequence
  A cell penetrading peptide
MVTVLFRRLRIRRACGPPRVRV

SEQ ID NO: 6 - protein, 21 aa, Artificial Sequence
  A cell penetrating peptide
KETWWETWWTEWSQPKKKRKV

SEQ ID NO: 7 - protein, 27 aa, Artificial Sequence
  A cell penetrating peptide
GWTKNSAGYLLGKINLKALAALAKKIL

SEQ ID NO: 8 - protein, 18 aa, Artificial Sequence
  Model Amphipathic Peptide
KLALKLALKALKAALKLA